(19) United States
(12) Patent Application Publication
(10) Pub. No.: US 2024/0319093 A1
(43) Pub. Date: Sep. 26, 2024

(54) SENSING CHIP, SENSING CHIP MANUFACTURING METHOD, SENSING KIT, MEASURING METHOD AND MEASURING DEVICE

(71) Applicant: KWANSEI GAKUIN EDUCATIONAL FOUNDATION, Hyogo (JP)

(72) Inventor: Keiko TAWA, Sanda-shi (JP)

(21) Appl. No.: 18/276,786

(22) PCT Filed: Jan. 28, 2022
(86) PCT No.: PCT/JP2022/003209

§ 371 Date: Aug. 10, 2023

(30) Foreign Application Priority Data
Feb. 26, 2021 (JP) 2021-029652

(51) Int. Cl. G01N 21/552; G01N 21/64

(52) U.S. Cl. CPC G01N 21/553; G01N 21/648

(57) ABSTRACT

A sensing chip includes a substrate having a plasmon-generating area of a periodic structure, and a plurality of capturing molecules for capturing a target substance. The plurality of capturing molecules is bonded to the plasmon-generating area at a higher density than to the area surrounding the plasmon-generating area. Thus, detection sensitivity in fluorescent observation can be improved.

### TECHNICAL FIELD

The present invention relates to a sensing chip, a sensing chip manufacturing method, a sensing kit, a measuring method and a measuring device that detect target substance by utilizing interaction of surface plasmon resonance. The present application claims convention priority on Japanese Patent Application No. 2021-029652 filed on Feb. 26, 2021, the entire contents of the Japanese patent application are hereby incorporated by reference.

### BACKGROUND ΔRT

Attaining higher sensitivity of biosensors, immunosensors and the like is required for various targets. Particularly, recent increase of new infectious diseases is fueling demand for highly sensitive immunosensor chips that can quickly detect markers of various diseases in a simple manner. In order to attain higher sensitivity, developments of high-affinity antibodies and improvement of signal-to-noise ratio are in progress.

A sensor chip capable of fluorescence enhancement utilizing surface plasmon resonance (hereinafter referred to as plasmonic chip) has been known as a tool for enhancing signal intensity. For example, Non-Patent Literature 1 discloses a Bull's eye type chip, comprising concentric circles whose cross-section passing through the center has a periodic structure. Specifically, it is disclosed that the Bull's eye structure enables illumination light having all azimuthal components from an objective lens to efficiently couple with surface plasmon (hereinafter referred to as plasmon) and to form an enhanced electric field under a microscope.

### CITATION LIST

### Non-Patent Literature

NPL 1: Mai Kanda, Eri Fujimoto, Keiko Tawa, “Evaluation of Fluorescence Enhancement of Nanoparticles on the Bull's eye-type Plasmonic Chip by Fluorescence Microscopy” Proceedings of the 67th JSAP Spring Meeting (the Japan Society of Applied Physics) (issued on Feb. 28, 2020)
NPL2: “Light sources and conditions for photoactivation of aryl azide crosslinking and labeling reagents”, [online], Thermo Fisher Scientific Inc. [Searched on Feb. 1, 2021] Internet <URL: https://www.thermofisher.com/document-connect/document-connect.html?url=https %3A %2F %2Fassets.thermofisher.com %2FTFS-Assets %2FLSG %2FApplication-Notes %2FTR0011-Photoactivate-aryl-azides.pdf&title=VGVjaCBUaXA6IExpZ2h0IHNvdXJjZXMgYW5kIGNvbmRpdGlvb nMgZm9yIHBob>
NPL3: Florian Feist, et al., “Visible Light-Induced Ligation via α-Quinodimethane Thioethers”, J. Am. Chem. Soc. 2018, 140, 37, 11848-11854

### SUMMARY OF INVENTION

### Technical Problem

By using plasmonic chips, particularly those having the Bull's eye structure, it becomes possible to detect enhanced signals of fluorescent labelling molecules in an optical system (microscope and the like) that irradiates and detects using an objective lens. Therefore, sensitivities of biosensors and immunosensors can be improved. However, still higher sensitivity is desired. Through intensive study, the inventor found an approach for improving sensitivity different from the method of enhancing signals of fluorescent labelling molecules. Specifically, the inventor conceived an idea of bonding capturing molecules for trapping a target substance such as antigens with a chip in position-selective manner (specifically, spatially controlling fixation of capturing molecules on the chip). If capturing molecules are successfully bonded to the chip in position-selective manner, it is expected to attain higher sensitivity of sensors.

Therefore, an object of the present invention is to provide a sensing chip, a sensing chip manufacturing method, a sensing kit as well as a measuring method and a measuring device, in which capturing molecules that capture target substance are bonded in position-selective manner.

### Solution to Problem

(1) According to a first aspect, the present invention provides a sensing chip, including: a substrate having a plasmon-generating area; and a plurality of capturing molecules for capturing a target substance; wherein the plurality of capturing molecules is bonded to the plasmon-generating area at a higher density than to the area surrounding the plasmon-generating area.

(2) Preferably, the plurality of capturing molecules is bonded to the central portion of the plasmon-generating area at a higher density than to the area surrounding the central portion.

(3) More preferably, the plasmon-generating area has a concentric periodic structure of projections and recesses.

(4) More preferably, a prescribed portion including the center of the concentric circles has a projected or recessed shape, and the prescribed portion is a circle of which diameter is at most one period of the structure of projections and recesses.

(5) Preferably, the capturing molecule includes biotin; and the biotin is bonded to the plasmon-generating area by a compound of maleimide, a compound represented by general formula (1) or (2) below or TFPA-PEG3-Biotin, and 3-Aminopropyl triethoxysilane. Therefore, it becomes possible to detect protein as the target substance with high density.

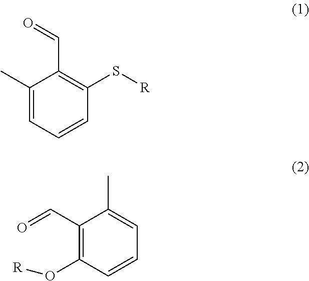

In the general formulae (1) and (2), R is any of the following compounds A1 to A11.

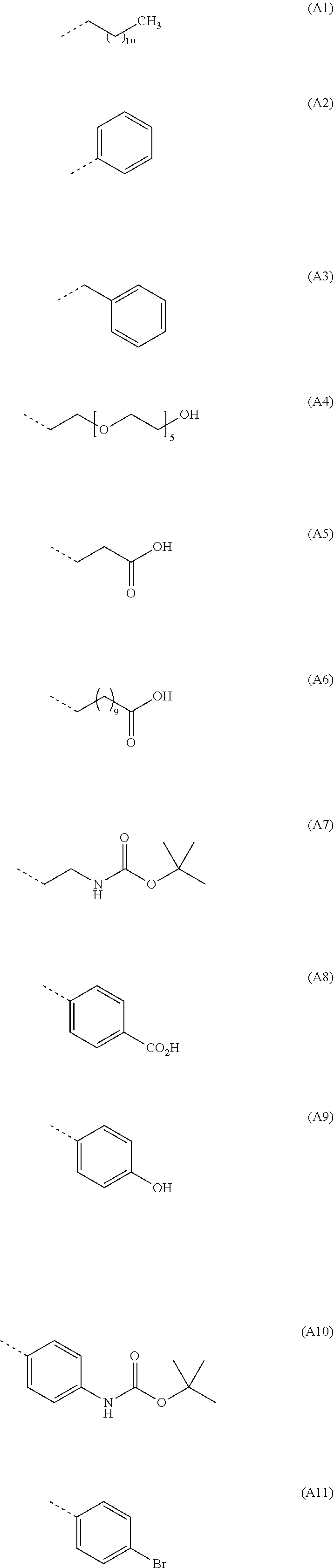

(6) According to a second aspect, the present invention provides a method of manufacturing a sensing chip, including: the first step of introducing a photoreaction compound bonded with capturing molecules for capturing a target substance, to a substrate having a plasmon-generating area; and the second step of irradiating light at a back surface of the substrate after execution of the first step; wherein at the second step, photoreaction of the photoreaction compound is promoted by plasmon-enhanced electric field, to have the capturing molecules bonded to the plasmon-generating area. Therefore, it becomes possible to selectively bond the capturing molecules with the plasmon-generating area, and hence, it becomes possible to detect the target substance with high sensitivity.

(7) According to a third aspect, the present invention provides a method of manufacturing a sensing chip, including: the first step of introducing capturing molecules for capturing a target substance to a substrate having a plasmon-generating area and having a photoreaction compound coupled; and the second step of irradiating light at a back surface of the substrate after execution of the first step; wherein at the second step, photoreaction of the photoreaction compound is promoted by plasmon-enhanced electric field, to have the capturing molecules bonded to the plasmon-generating area. Therefore, it becomes possible to selectively bond the capturing molecules with the plasmon-generating area, and hence, it becomes possible to detect the target substance with high sensitivity.

(8) Preferably, the light irradiated at the second step has a wavelength of at least 300 nm and at most 550 nm, or at least 600 nm and at most 1100 nm.

(9) More preferably, the photoreaction compound includes a compound represented by general formula (1) or (2) below, or TFPA-PEG3-Biotin. Thus, it becomes possible to bond the capturing molecules with the plasmon-generating area at a higher density as compared with the area surrounding the plasmon-generating area, and therefore it becomes possible to detect the target substance with higher sensitivity.

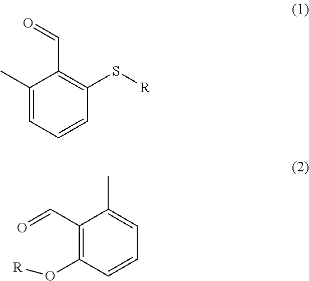

In the general formulae (1) and (2), R is any of the following compounds A1 to A11.

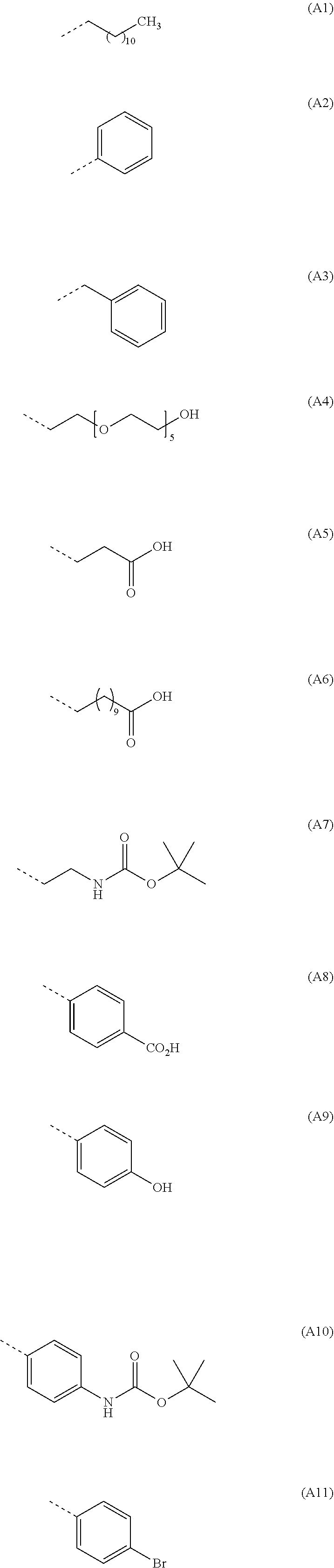

(10) More preferably, the light irradiated at the second step has a wavelength of at least 450 nm and at most 490 nm. Thus, it is possible to suppress bonding of capturing molecules outside the plasmon-generating area of the chip. Further, it is possible to bond a larger number of capturing molecules at the center than the periphery of the plasmon-generating area. Therefore, the target substance can be detected with still higher sensitivity.

(11) According to a fourth aspect, the present invention provides a sensing kit, including a substrate having a plasmon-generating area and a photoreaction compound; wherein by introducing the photoreaction compound to the substrate and irradiating light at the back surface of the substrate, photoreaction of the photoreaction compound is promoted by a plasmon-enhanced electric field, and the photoreaction compound comes to be bonded to the plasmon-generating area at a higher density than to the area surrounding the plasmon-generating area.

(12) According to a fifth aspect, the present invention provides a measuring method, including: the first step of introducing, to the above-described sensing chip, the target substance to which the fluorescent substance is coupled; and the second step of irradiating the sensing chip at its back surface with light after execution of the first step, and measuring fluorescent light emitted from the fluorescent substance by a plasmon-enhanced electric field, from the front surface of the sensing chip.

(13) According to a sixth aspect, the present invention provides a measuring device, including a light source, and a lens for collecting light from the light source; wherein in a state where a photoreaction compound bonded with capturing molecules for capturing a target substance is introduced to a substrate having a plasmon-generating area, the substrate is irradiated at its back surface with the light collected by the lens, whereby photoreaction of the photoreaction compound is promoted by a plasmon-enhanced electric field and the capturing molecules are bonded to the plasmon-generating area. The device further includes a measuring unit for measuring fluorescent light emitted from a fluorescent substance by a plasmon-enhanced electric field, after irradiating the substrate at its back surface with the light collected by the lens in a state where the target substance containing the fluorescent substance is introduced to the substrate having the capturing molecules bonded to the plasmon-generating area.

### Effects of Invention

According to the present invention, by using a sensing chip having the capturing molecules bonded in a position-selective manner, detection sensitivity in fluorescent observation can be enhanced than before. Therefore, by using the sensing chip of the invention as a biosensor or an immunosensor, a highly-sensitive measurement device that can detect markers of each disease in a simple and quick manner can be realized. Further, of the propagating plasmons generated in the plasmon-generating area, grating-coupled surface plasmon resonance enhances electromagnetic field strength, resulting in the degree of enhancement depending on the grating structure. Thus, a sensing chip that promotes optical response (that is, photochemical reaction) and having capturing molecules bonded in a position-selective manner can efficiently be manufactured.

Further, in one same chip, it is possible to easily fabricate an area where the capturing molecules are bonded and the other area where not, and using the fluorescent intensity in the area free of capturing molecules as a base intensity, original signal strength can be evaluated with high accuracy. Further, different from the manufacturing method locally causing photo-reaction by using a mask, the manufacturing method of the present invention makes unnecessary the troublesome steps of mounting (including registration) a mask on the chip and removing the mask after reaction, and hence, the process for manufacturing the sensing chips can be simplified.

### BRIEF DESCRIPTION OF DRAWINGS

FIG. 1 is a plan view schematically showing a structure of a sensing chip in accordance with an embodiment of the present invention.

FIG. 2 shows an AFM (Atomic Force Microscope) image showing a concentric Bull's eye structure as an example of the plasmon-forming area.

FIG. 3 is a cross-section showing the structure of the sensing chip shown in FIG. 1.

FIG. 4 shows the chemical formula of APTES (3-Aminopropyl triethoxysilane).

FIG. 5 shows a state of chip body surface (SiO₂) modified with APTES.

FIG. 6. shows the chemical formula of o-Methylbenzaldehydes as an example of photoreaction compound.

FIG. 7 shows the chip body in the state of FIG. 5, coupled with o-Methylbenzaldehydes shown in FIG. 6.

FIG. 8 shows the chip body in the state of FIG. 7 to which maleimide compound is introduced.

FIG. 9 shows a state in which a photoreaction compound and maleimide compound are coupled to each other.

FIG. 10 shows another method of manufacturing the sensing chip.

FIG. 11 shows chemical formula of Succinimidyl PEG.

FIG. 12 is a block diagram showing a schematic structure of the measuring device.

FIG. 13 is a plan view showing an example of a periodic structure of plasmon-forming area different from the Bull's eye structure.

FIG. 14 is a plan view showing an example of a periodic structure of plasmon-forming area different from FIG. 13.

FIG. 15 shows the chemical formula of TFPA-PEG3-Biotin as an example of photoreaction compound.

FIG. 16 shows photo-reaction of TFPA-PEG3-Biotin shown in FIG. 15.

FIG. 17 is a plan view showing a structure of a prototype model sensing chip.

FIG. 18 is a photograph showing experimental result.

FIG. 19 shows a state of a substrate coupled with biotin-maleimide including capturing molecules to which Cy5-streptavidin is introduced as a target substance, in Example 3.

FIG. 20 shows a state in which the introduced Cy5-streptavidin is bonded with biotin-maleimide coupled with the substrate.

FIG. 21 shows a result of fluorescent observation, using a chip fabricated by UV irradiation to cause photoreaction, in Example 3.

FIG. 22 shows a result of fluorescent observation, using a chip fabricated under conditions different from those for the chip giving the result shown in FIG. 21, by UV irradiation to cause photoreaction, in Example 3.

FIG. 23 shows a result of fluorescent observation, without UV irradiation to the chip to cause photoreaction, as Comparative Example.

FIG. 24 shows a result of fluorescent observation, after irradiating the chip with visible light to cause photoreaction, in Example 4.

FIG. 25 is a photograph showing a result of fluorescent observation, using a chip fabricated without light irradiation to promote photoreaction and without introducing Cy5-maleimide.

FIG. 26 is a photograph showing a result of fluorescent observation, using a chip fabricated without light irradiation to promote photoreaction but introducing Cy5-maleimide.

FIG. 27 is a photograph showing a result of fluorescent observation, using a chip fabricated by visible light irradiation to promote photoreaction and introducing Cy5-maleimide, in Example 5.

### DESCRIPTION OF EMBODIMENTS

In the following embodiments, the same components are denoted by the same reference characters. Their names and functions are also the same. Therefore, detailed description thereof will not be repeated.

### (Sensing Chip Structure)

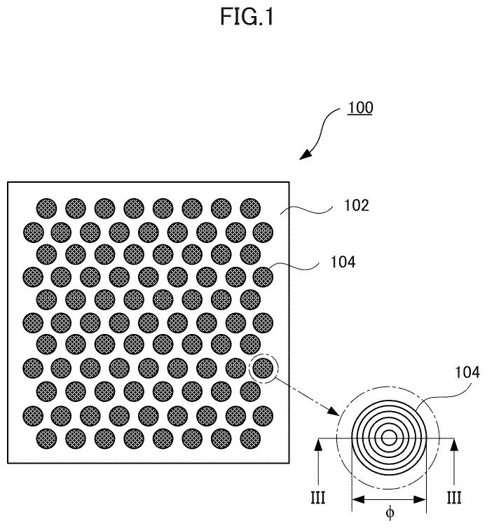

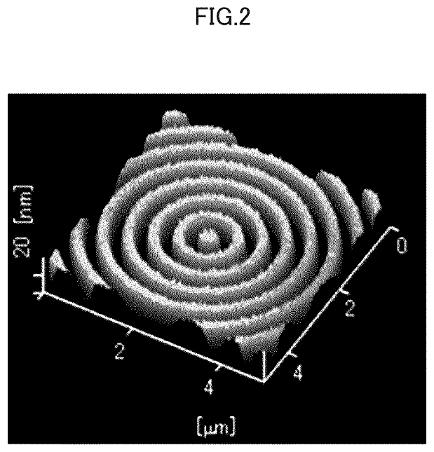

Referring to FIG. 1, a sensing chip 100 in accordance with an embodiment of the present invention includes a chip body 102 and a plasmon-generating area 104 formed on chip body 102. In FIG. 1, a plurality of plasmon-generating areas 104 are arranged in a hexagonal lattice. On the lower right side of FIG. 1, one plasmon-generating area 104 is shown in enlargement. Here, as plasmon-generating area 104, a Bull's eye structure (see AFM image of FIG. 2) is used, which has periodic recesses and projections formed concentrically in a circular area having the diameter of ϕ.

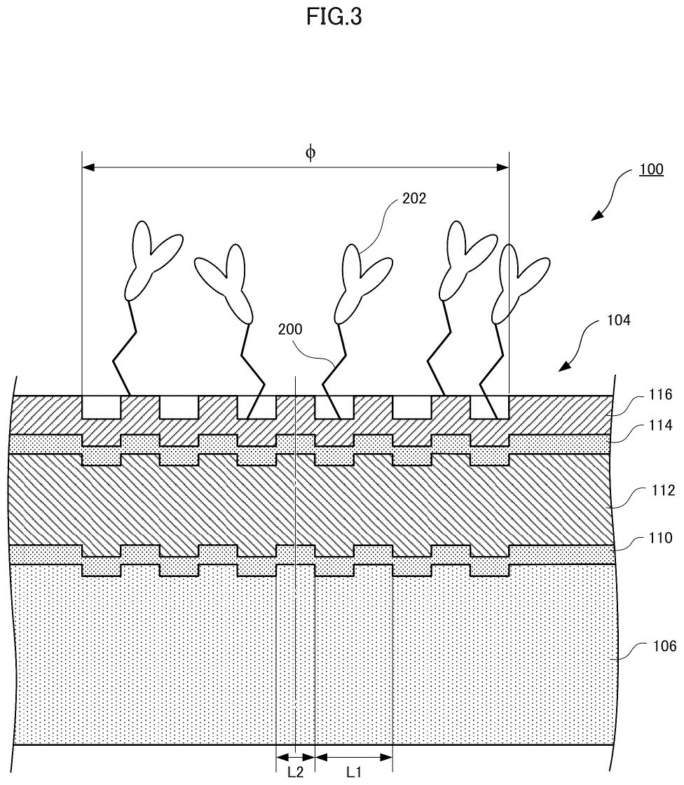

FIG. 3 is a cross-section of the plasmon-generating area 104 shown at the lower right side of FIG. 1, taken along the line III-III passing through its center. Referring to FIG. 3, plasmon-generating area 104 includes a base substrate 106 having the above-described periodic structure (that is, Bull's eye structure), and a multi-layered film including a first adhesive layer 110, a metal layer 112, a second adhesive layer 114 and a quench-suppressing layer 116, formed on the base substrate 106. Plasmon-generating area 104 further includes a coupled compound 200 arranged on the multi-layered film, and capturing molecule 202 bonded to coupled compound 200. Coupled compound 200 refers to a compound in which a plurality of substances is coupled by photoreaction (that is, photochemical reaction) using enhanced electric field caused by plasmon resonance, which will be described later. Though capturing molecules 202 may exist on areas surrounding plasmon-generating area 104, they exist unevenly on plasmon-generating area 104. In other words, capturing molecules 202 are bonded to plasmon-generating area 104 at a higher density than to the area around plasmon-generating area 104.

Base substrate 106 is formed, for example, of glass, plastic (e.g. polymethylmethacrylate (PMMA)) or the like. Base substrate 106 may be transparent or non-transparent. The periodic structure may be formed by a known method (nano-print, press-molding or injection molding using a stamper, and so on).

In the Bull's eye structure, the period L1 (sum of adjacent recess and projection widths) of the concentric periodic structure is constant. In the Bull's eye structure shown in FIG. 3, the central portion has a protruding shape having a diameter L2 (a circle of which center is the center of concentric circles of the periodic structure). Typically used Bull's eye structures often have the central portion formed as a through hole penetrating the substrate. In plasmon-generating area 104, the central portion is not a through hole. The period L1 is preferably equal to or shorter than, or about the same as the wavelength of light used for fluorescent observation. For example, the period L1 is 100 to 1000 nm and, preferably, 200 to 600 nm.

In order to attain maximum fluorescence intensity, the diameter L2 of the central portion of Bull's eye structure should preferably be equal to half the period L1 as shown in FIG. 3. It may not necessarily be equal, as long as plasmons can be generated by light irradiation. For example, as will be described later as experimental results, the diameter L2 may be equal to or smaller than the period L1. The central portion of Bull's eye structure may have a recessed shape. The diameter L2 of the recess may or may not be equal to half the period L1.

The first adhesive layer 110 is for adhering base substrate 106 and metal layer 112. If base substrate 106 itself is of a material that stably fixes on metal layer 112, the first adhesive layer 110 may be omitted. The second adhesive layer 114 is for adhering metal layer 112 and quench-suppressing layer 116. If quench-suppressing layer 116 is of a material that stably fixes on metal layer 112, the second adhesive layer 114 may be omitted. Further, as will be described later, quench-suppressing layer 116 may be omitted. The first and second adhesive layers 110 and 114 are preferably as thin as possible and, by way of example, formed as a thin film of titanium (Ti) having the thickness of 0.1 to 3 nm. Use of titanium improves chip resistance to surfactant (Tween 20) included in PBS (phosphate buffer solution) used for cleaning in a bioassay and the like. The first adhesive layer 110 may be of chromium (Cr).

Metal layer 112 is, for example, silver (Ag) and formed by sputtering. Assuming irradiation at the back surface, the thickness of metal layer (Ag) 112 is preferably 10 to 100 nm and, more preferably, 30 to 65 nm. In FIG. 3, the shape of metal layer 112 is shown as having the same recesses and projections as base substrate 106. When metal layer 112 is formed, for example, by sputtering, the portions corresponding to the steps of the periodic structure of base substrate 106 come to be inclined. Therefore, the second adhesive layer 114 and the quench-suppressing layer 116, which will be described later, may also have shapes with inclinations.

Quench-suppressing layer 116 also serves as a layer for bonding capturing molecules (for example, antigen), and it is preferably formed of silicon dioxide (SiO₂), so as to allow use of a commercially available bioassay kit (for example, medical agent). Commercially available medical agents often assume application to SiO₂. SiO₂ does not absorb (or hardly absorb) the light of the wavelength range generally used as incident light and the fluorescent light emitted during observation and, therefore, it can be formed as a transparent thin film. Quench-suppressing layer 116 may be formed, for example, by sputtering.

If the distance between fluorescent molecules and metal layer 112 is close, even the fluorescent light excited by strong excitation field has its energy moved to the metal surface, and the enhanced fluorescence characteristic of Surface Plasmon-field enhanced Fluorescence Spectroscopy is quenched. Therefore, it is preferable to suppress quenching by separating the fluorescent molecules from metal layer 112 by a prescribed distance. For this purpose, if the molecule layers of coupled compound 200 and capturing molecules 202 shown in FIG. 3 have sufficient thicknesses, quench-suppressing layer 116 may be omitted. Further, excitation field of surface plasmon resonance is a near field and, therefore, the electric field strength thereof decays as it is further away from the metal surface. Therefore, only the fluorescent molecules existing within about 100 nm from the surface of metal layer 112 are excited efficiently. Therefore, the thickness of quench-suppressing layer 116 is determined in the range of about 10 nm to about 100 nm, considering the type of metal layer 112, refraction index of quench-suppressing layer 116, the wavelength of incident light and the like. The multi-layered film formed on the base substrate 106 may include a protective film in addition to those mentioned above. By way of example, when the protective film is arranged between metal layer 112 and quench-suppressing layer 116, the sum thickness of protective layer and quench-suppressing layer 116 should preferably be determined in the range of about 10 nm to about 100 nm, considering the type of metal layer 112, refraction index of quench-suppressing layer 116, the wavelength of incident light and the like.

Sensing chip 100 is used for detecting an antigen-antibody reaction. Preferably, the capturing molecule 202 is one that reacts to the antigen as the object of capturing. Specifically, it should preferably be the one that causes antigen-antibody reaction with the antigen to be captured. When a solution containing an antigen is dropped on sensing chip 100, an antigen-antibody reaction takes place between the input antigen and the capturing molecule 202 bonded through coupled compound 200 to the surface (that is, quench-suppressing layer 116) of plasmon-generating area 104. The antigen or the antibody (that is, capturing molecule 202) is coupled with fluorescent labelling protein (that is, fluorescent molecule) in advance. In this state, sensing chip 100 is irradiated with light at the back surface (that is, from the surface on which the periodic structure is not formed). Thus, surface plasmon resonance occurs in the plasmon-generating area 104, and enhanced fluorescence from the fluorescent labelling protein coupled to the antigen or antibody can be detected.

As will be described later, non-specific adherence of coupled compound 200 and capturing molecules 202 is sometimes observed around plasmon-generating area 104 and, therefore, fluorescent light is also emitted therefrom. While such fluorescence may be noise, it is negligible as compared with the enhanced fluorescence intensity emitted from plasmon-generating area 104. Therefore, sensing chip 100 enables highly sensitive detection.

### (Sensing Chip Manufacturing Method)

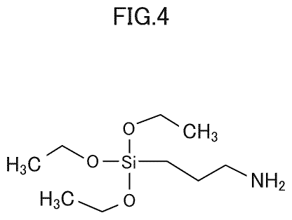

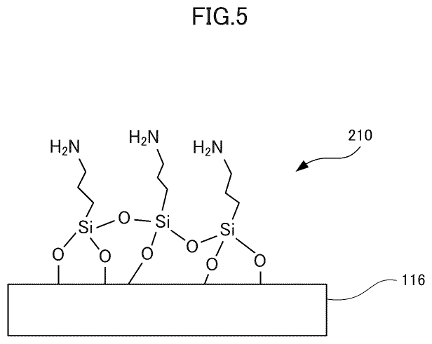

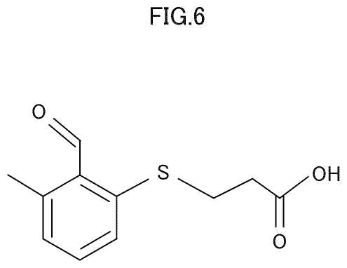

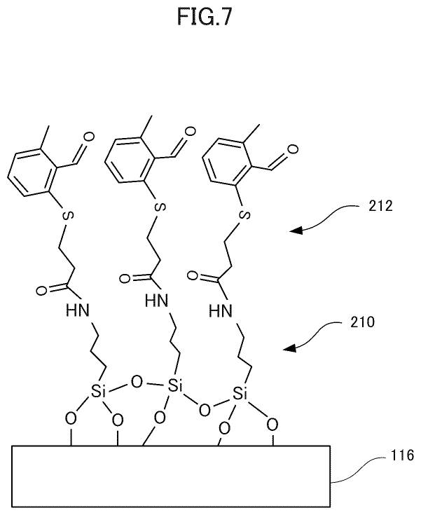

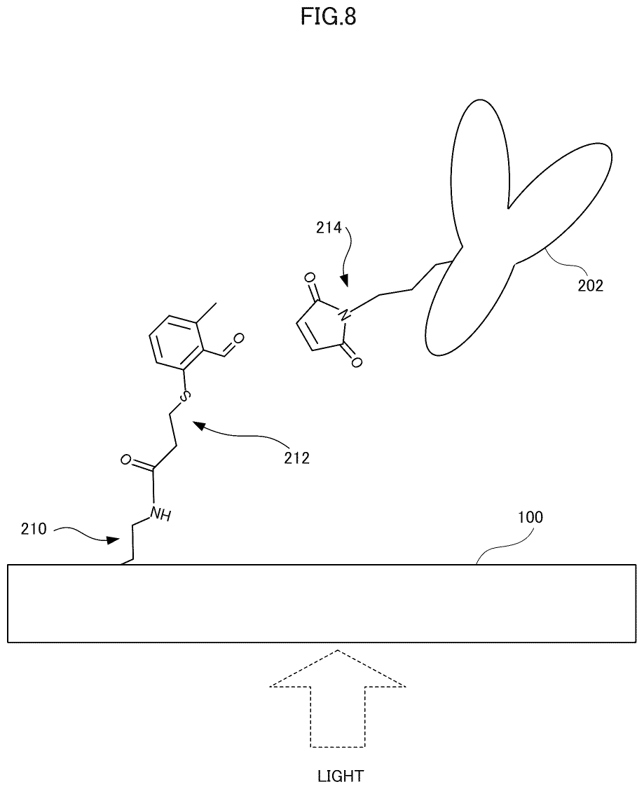

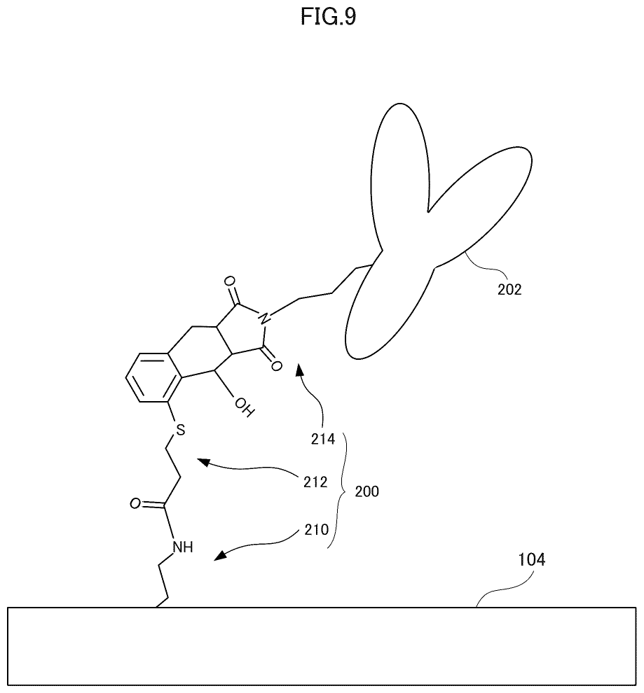

The method of manufacturing sensing chip 100 includes the following steps 1 to 4.
Step 1: On chip body 102, APTES (see FIG. 4) is positioned by silane coupling. Terminal end of APTES that is not coupled to the substrate is amino group. FIG. 5 shows a state in which the surface of quench-suppressing layer 116 on chip body 102 is silane-coupled with APTES 210.
Step 2: To the chip body 102 that has been subjected to Step 1, a photo-reaction compound is introduced, and the photoreaction compound is bonded to the amino group of APTES (amide bond). As the photoreaction compound, by way of example, o-Methylbenzaldehydes (hereinafter also referred to as benzaldehyde) shown in FIG. 6 is used. The photoreaction compound is introduced by preparing a DMF solution. Specifically, a solution is prepared by adding TEA (Triethylamine), EDC (1-Ethyl-3-(3-dimethylaminopropyl) carbodiimide Hydrochloride) and the photoreaction compound to DMF (N,N-dimethylformamide). This solution is put into the substrate on which Step 1 was performed, and is left standing still for a prescribed time period (for example, 2 hours). By dehydration reaction, APTES and o-Methylbenzaldehydes are amide-bonded as shown in FIG. 7.
Step 3: To the chip body 102 that has been subjected to Step 2, maleimide compound modified with the capturing molecule (antibody) is input as shown in FIG. 8 and the substrate is irradiated at the back surface with light (for example, ultraviolet light, hereinafter denoted as UV light) for a prescribed time period (for example, 5 minutes). FIG. 8 shows, as the maleimide compound, N-succinimidyl-3-maleimidepropionate 214 modified with capturing molecule 202. By light irradiation, a plasmon-enhanced electric field is formed in plasmon-generating area 104. This promotes photoreaction of the photoreaction compound (that is, benzaldehyde 212) and the maleimide compound (that is, N-succinimidyl-3-maleimidepropionate 214). As a result, as shown in FIG. 9, the photoreaction compound (that is, benzaldehyde 212) and the maleimide compound (that is, N-succinimidyl-3-maleimidepropionate 214) are coupled in the plasmon-generating area 104 and, thus, coupled compound 200 comes to be concentratively coupled to the plasmon-generating area 104. FIG. 9 shows the state in which o-Methylbenzaldehydes group is coupled with maleimide group.
Step 4: After Step 3, chip body 102 is rinsed. As the cleaning liquid, mixed phosphate buffer solution (that is, Tween 20), which is an example of interfacial active agents, may be used. At Step 3, in the area surrounding plasmon-generating area 104, photoreaction between the photoreaction compound (that is, benzaldehyde 212) and the maleimide compound (that is, N-succinimidyl-3-maleimidepropionate 214) is not promoted. Not-yet-reacted maleimide compound (that is, N-succinimidyl-3-maleimidepropionate 214) is removed from the chip body 102 by rinsing.

Through the process described above, the sensing chip 100 having the capturing molecules 202 bonded to specific areas (that is, plasmon-generating area 104) of chip body 102 in position-selective manner is realized. Specifically, in the sensing chip 100, the capturing molecules 202 are bonded to the plasmon-generating area 104 at a higher density than to the surrounding area surrounding plasmon-generating area 104. The characteristic point is that by irradiating light, photoreaction is promoted using the plasmon-enhanced electric field in the plasmon-generating area 104, and that capturing antibody is bonded concentratively in the plasmon-generating area 104. This leads to a significant effect of enhanced fluorescence, as will be described later. Since bonding of capturing antibody is suppressed in the areas surrounding plasmon-generating area 104, detection sensitivity improves.

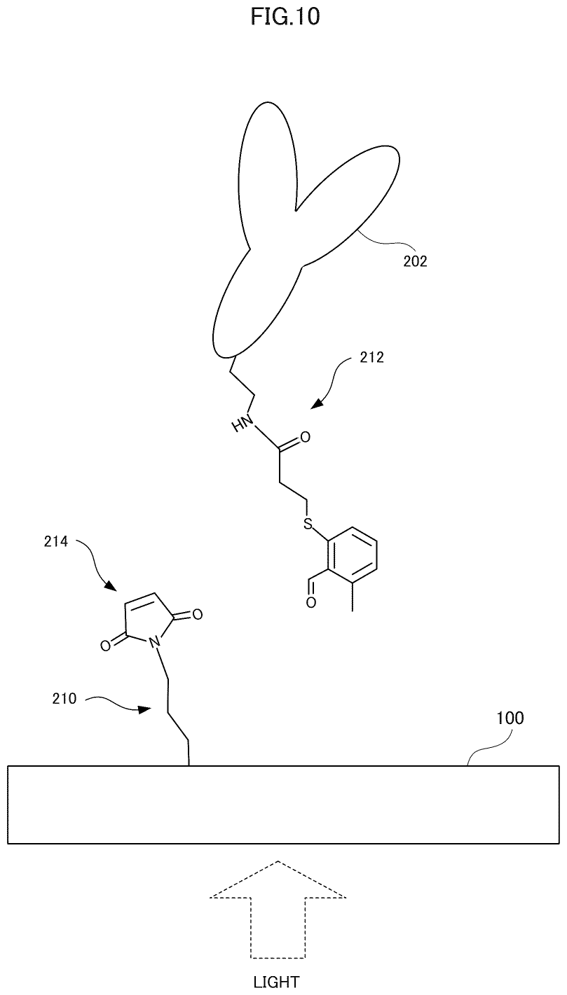

In the above-described manufacturing method, while chip body 102 is coupled with the compound of APTES 210 and benzaldehyde 212, N-succinimidyl-3-maleimidepropionate 214 modified with capturing molecule 202 is introduced, and the chemical reaction is promoted by plasmon resonance. It is, however, not limiting. As shown in FIG. 10, while the chip body 102 is coupled with the compound of APTES 210 and N-succinimidyl-3-maleimidepropionate 214, photoreaction compound (that is, benzaldehyde 212) modified with capturing molecule 202 may be introduced thereto, followed by UV light irradiation. Then, a plasmon-enhanced electric field is formed in the plasmon-generating area 104, promoting photoreaction of photoreaction compound (that is, benzaldehyde 212) and the maleimide compound (that is, N-succinimidyl-3-maleimidepropionate 214) is promoted. Therefore, as in the example of FIG. 9, the photoreaction compound (that is, benzaldehyde 212) and the maleimide compound (that is, N-succinimidyl-3-maleimidepropionate 214) are coupled in the plasmon-generating area 104, and a sensing chip 100 having capturing molecules 202 bonded in a position-selective manner to the specific area (that is, plasmon-generating area 104) of chip body 102 is thus fabricated.

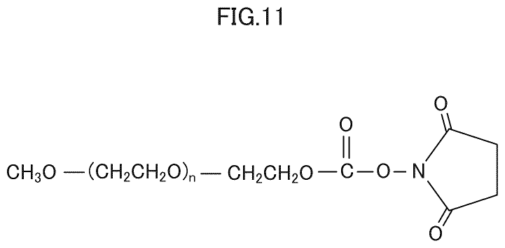

Further, at Step 2 above, the photoreaction compound (that is, benzaldehyde 212) may be mixed with Succinimidyl PEG, of which terminal end is a carboxyl group, shown in FIG. 11, and the mixture may be input to chip body 102 that has been subjected to Step 1. The maleimide compound modified with the capturing molecule introduced at Step 3 may possibly be adhered non-specifically to chip body 102, in addition to coupling with photoreaction compound. When antigen with fluorescent labelling is put into the chip as such, antigen-antibody reaction occurs with the capturing molecule (for example, antibody) non-specifically adhered to the chip body 102 and fluorescent light comes to be emitted also from around the plasmon-generating area 104. This leads to lower detection sensitivity. Introduction of Succinimidyl PEG is expected to be effective at step 3 in preventing non-specific adhesion of maleimide compound modified with the capturing molecule to the chip body 102.

If a localized plasmon-enhanced electric field can be formed at a portion of plasmon-generating area 104, it can be expected that the above-described photoreaction is concentratedly promoted at that portion of plasmon-generating area 104. The Bull's eye structure adopted in the plasmon-generating area 104 enables formation of the localized plasmon-enhanced electric field, by the optical antenna effect at the central portion of the Bull's eye structure. Accordingly, the capturing molecules can be bonded to the central portion of plasmon-generating area 104 at a higher density than to the surrounding area. In order to bond the capturing molecules at a high density to the central portion of plasmon-generating area 104, it is preferred to irradiate the plasmon-generating area 104 with light in the visible range (380 nm to 780 nm), for example, in place of UV light. Generally, the effect of promoting photoreaction is small when the light of the visible range is irradiated. In contrast, irradiation of light in the visible range to the plasmon-generating area 104 realizes local existence of plasmons at the central portion of plasmon-generating area 104, and photoreaction can be promoted concentratedly at the central portion of plasmon-generating area 104. By way of example, a solution containing minute spheres (such as silica beads whose diameter is at most 1 μm) of which surface is modified with capturing antibody, biotin compound or streptavidin coupled with capturing antibody or the like is introduced to the chip body 102, followed by irradiation of visible light of 300 nm to 550 nm or 600 nm to 1100 nm. In this manner, silica beads or biotin compound can be coupled concentratedly at the central portion of plasmon-generating area 104. When visible light of 300 nm to 550 nm (for example, 450 nm) is used, photoreaction of one photon can be promoted, and when visible light of 600 nm to 1100 nm (for example, 720 nm) is used, photoreaction by two photons can be promoted. By using the sensing chip fabricated in this manner, fluorescent observation of antigen-antibody reaction with still higher sensitivity becomes possible, by introducing, for example, fluorescent-labelled antigen.

The chip body 102 having the above-described plasmon-generating area 104 can form, combined with a photoreaction compound, a sensing kit. Using this kit, by inputting a photoreaction compound to the chip 102 and irradiating with light, photoreaction can be promoted by plasmon-enhanced electric field, and the photoreaction compound can be coupled in a concentrated manner in the plasmon-generating area 104.

### (Measuring Device)

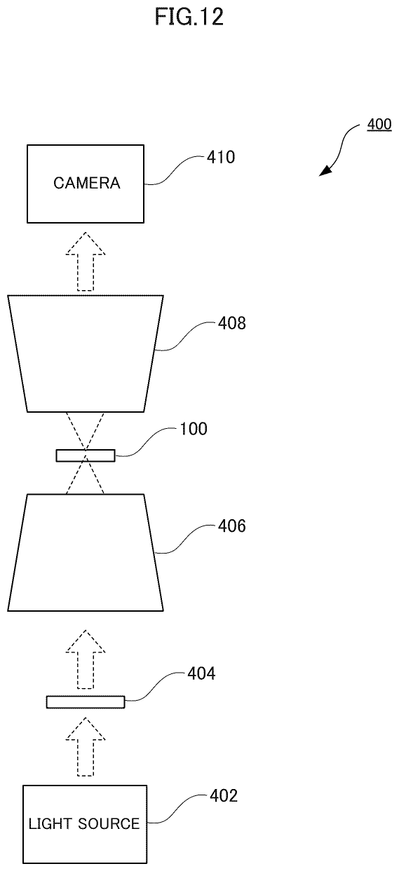

A device used for manufacturing the sensing chip 100 and for fluorescent observation using the sensing chip 100 will be described. Referring to FIG. 12, a measuring device 400 includes the sensing chip 100, a light source 402, an optical filter 404, a first lens 406, a second lens 408 and a camera 410. When the measuring device 400 is used for manufacturing the sensing chip 100, the sensing chip 100 shown in FIG. 12 is replaced by a chip body 102 not modified with capturing molecules 202.

Light source 402 is a mercury lamp or a halogen lamp. Optical filter 404 passes light of a specific wavelength of the light emitted from light source 402 and blocks others. As optical filter 404, by way of example, a Cy5 filter (that is, a bandpass filter that passes excitation light of fluorescent substance Cy5) or a NUA filter (that is, a bandpass filer that passes the wavelength of 370 nm to 380 nm) may be used.

The first lens 406 is an objective lens for collecting light that has passed through optical filter 404. For fluorescent observation using sensing chip 100, an objective lens having a 20× magnification, for example, is used as the first lens 406. At this time, a halogen lamp is used as light source 402, and if the sensing chip 100 is modified with molecules fluorescent-labelled with Cy5, for example, a Cy5 filter is used as optical filter 404. When Step 3 described above is to be executed by irradiating light from optical filter 404 to the back surface of chip body 102 by using chip body 102 in place of sensing chip 100, an objective lens having a 100× magnification is used as the first lens 406. At this time, a mercury lamp is used as light source 402 and an NUA filter is used as optical filter 404.

The second lens 408 is for collecting light emitted from sensing chip 100 and outputting to camera 410. The second lens 408 is, for example, a lens having a 10× magnification. Camera 410 is an imaging device (for example, a CCD camera). The measuring device 400 may include an optical system (such as a prism, a mirror and the like) other than the components shown in FIG. 12.

By using the sensing chip 100 described above, local photoreaction within a pattern can be promoted in the plasmon-generating area 104 having the concentric periodic structure. As to the photoreaction, the photoreaction between a compound having maleimide group and the photoreaction compound can be realized with light in the wavelength range from UV to visible light. Particularly, in the near infrared range, 2-photon reaction is expected. Further, in the concentric structure, a strong electric field is formed particularly at the central portion and, therefore, in the pattern, a local electric field can be formed particularly at the central portion. The local photoreaction is expected to realize detection with high sensitivity in establishing an immunoassay.

In the foregoing, an example has been described in which a light source (mercury lamp) for promoting photoreaction is different from a light source (halogen lamp) at the time of observing enhanced fluorescence. The foregoing, however, is not limiting. Promotion of photoreaction and observation of enhanced fluorescence may be executed by using a single light source including in its emission range both the wavelength for promoting photoreaction and the wavelength for fluorescent observation.

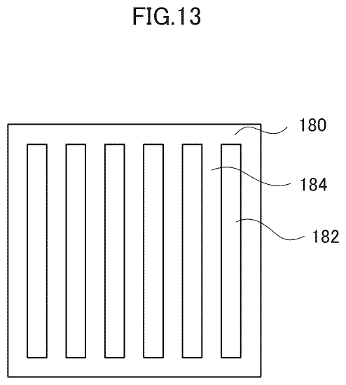

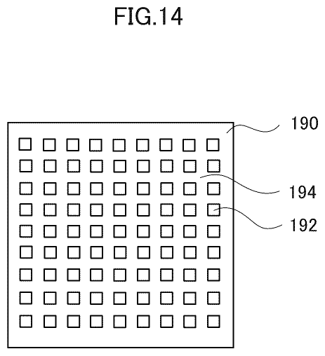

Though an example has been described in which the periodic structure of plasmon-generating area 104 is a Bull's eye structure in the foregoing, it is not limiting. The periodic structure of plasmon-generating area 104 may have periodic parallel recesses and projections formed in a direction as shown in FIG. 13 (that is, line & space pattern). In FIG. 13, projections 182 are formed parallel to each other in one direction on the surface of base substrate 180, and recesses 184 are formed around projections 182. The structure may also be a two-dimensional periodic structure such as shown in FIG. 14. In FIG. 14, projections 192 are formed in two intersecting directions on a surface of a base substrate 190, and recesses 194 are formed around projections 192. Further, a structure having recesses and projections of FIG. 13 reversed, or a Hole Array having recesses and projections of FIG. 14 reversed, may be used.

The cross-sectional form of the recess (trench) in the periodic structure of plasmon-generating area 104 is not limited to the rectangular shape shown in FIG. 3, and it may have a sawtooth shape or a sinusoidal wave shape.

Metal layer 112 is not limited to silver (Ag) and any metal that causes surface plasmon resonance may be used. Metal layer 112 may be of gold (Au), aluminum (A1), etc.

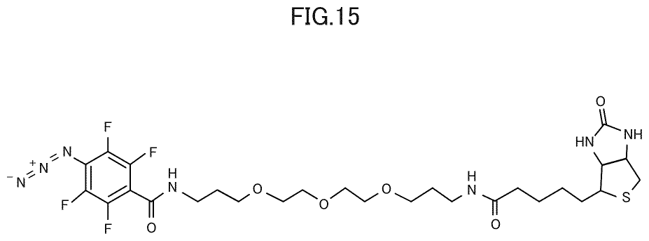

Though o-Methylbenzaldehydes (see FIG. 6) is shown as a photoreaction compound, it is not limiting. A commercially available reagent may be used to prepare the interface through photoreaction with APTES surface. By way of example, TFPA-PEG3-Biotin shown in FIG. 15 may be used as a photoreaction compound. In that case, as a reaction compound replacing maleimide compound (N-succinimidyl-3-maleimidepropionate 214), use of an avidin-modified antibody, or an avidin-biotin-modified antibody is preferable.

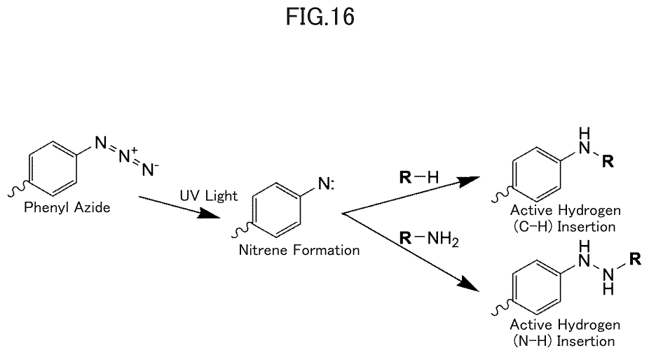

To the chip body 102 having APTES 210 coupled to its surface, TFPA-PEG3-Biotin is introduced, and it is irradiated with UV light. Thus, in the plasmon-generating area 104, photoreaction shown in FIG. 16 (see Non-Patent Literature 2) is promoted by the plasmon-enhanced electric field. Therefore, it becomes possible to couple TFPA-PEG3-Biotin in a concentrated manner to APTES 210 coupled to the plasmon-generating area 104. Thereafter, an avidin-modified antibody or an avidin-biotin-modified antibody is bonded to TFPA-PEG3-Biotin, and thus, the sensing chip 100 having the structure shown in FIG. 3 can be formed.

Further, as a photoreaction compound, a compound represented by general formula (1) or (2) below may be used.

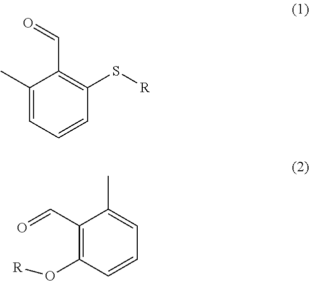

In the general formulae (1) and (2), R is any of the following compounds A1 to A11. When R is A5, general formula (1) represents 3-((2-formyl-3-methylphenyl)thio) propanoic acid, that is, o-Methylbenzaldehydes shown in FIG. 6.

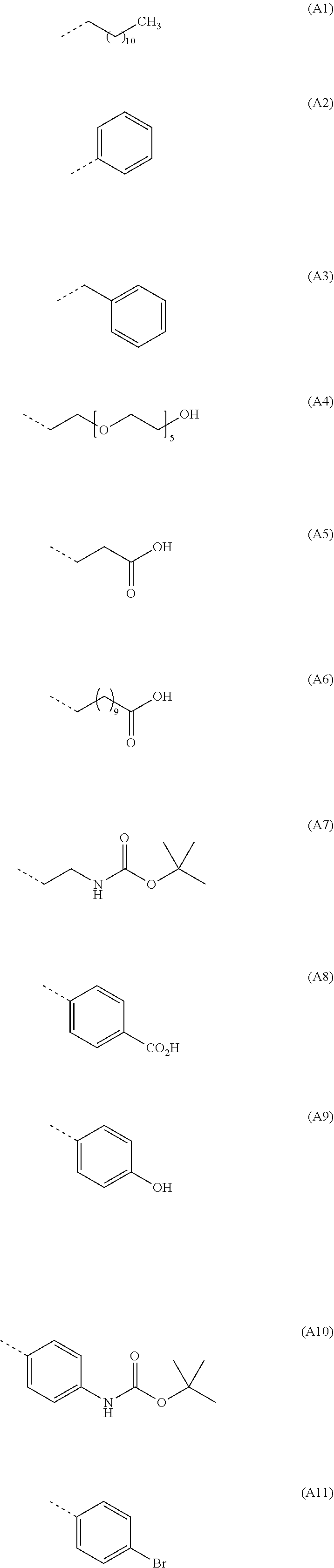

The compound represented by general formulae (1) and (2) (R is any of A1 to A11) can be prepared by the method disclosed in Non-Patent Literature 3.

The coupled compound 200 that bonds capturing molecule 202 to chip body 102 is not limited to one containing APTES 210 coupled to quench-suppressing layer 116. Coupled compound 200 is formed by photoreaction of photoreaction compound during the process of manufacturing sensing chip 100, and it may be any substance that is coupled to quench-suppressing layer 116. By the photoreaction of photoreaction compound, it becomes possible to concentratedly couple coupled compound 200 to the plasmon-generating area 104 and, hence, to bond the capturing molecules 202. Further, the object (target substance) captured by capturing molecules 202 is not limited to an antibody and, it may be, for example, DNA. The capturing molecules 202 may be any molecule that captures the target substance. For example, the capturing molecule 202 may be a compound having a portion that attains specific absorption to the target substance.

### Example 1

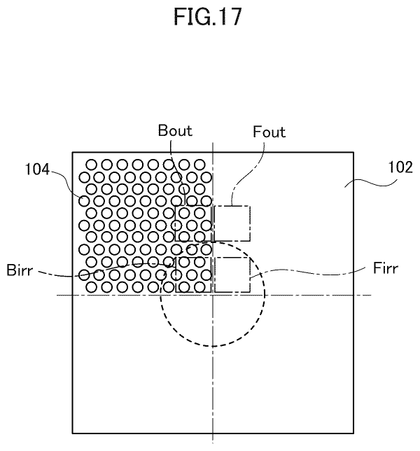

In the following, experimental results will be described to show effectiveness of the present invention. A chip having the structure shown in FIG. 17 was prepared by way of trial. Only on the upper left area of chip body 102, approximately 2000 plasmon-generating areas 104 were formed. The plasmon-generating area 104 has an outer diameter of 20 μm and a period of 480 nm (that is, the distance between adjacent projections is 240 nm), and the central portion has a projected shape having the diameter of 480 nm. The plurality of plasmon-generating areas 104 is arranged in a hexagonal lattice, apart from each other by the distance of 5 μm (therefore, the center-to-center distance is 25 μm). On the base substrate on which such plasmon-generating areas 104 were formed, the above-described multi-layered film was formed. Specifically, the first and second layers were formed by using Ti, each to the thickness of under 1 nm, the metal layer was formed by using Ag to the thickness of 45 nm, and the quench-suppressing layer was formed by using SiO₂ to the thickness of 20 nm.

The above-described chip body 102 having the plasmon-generating areas 104 formed thereon was prepared, and a chip modified by compounds in the same manner as the manufacturing method described above was fabricated. Specifically, APTES (see FIG. 5) was coupled to the chip body, and o-Methylbenzaldehydes (see FIG. 6) was introduced as photoreaction compound and left still-standing for 2 hours, to attain coupling shown in FIG. 7. For preparing a DMF solution, 2 mL of DMF, 15 μL of TEA, 11.5 mg of EDC and 11.2 mg of o-Methylbenzaldehydes were used. These processes were done in a darkroom or in a room with yellow lamp. Thereafter, 3.12 nM of Cy5-maleimide, which is a maleimide compound and also a fluorescent substance, was introduced and then, chip body 102 was irradiated at the back surface with UV light to promote photoreaction of o-Methylbenzaldehydes. As the light source and as the optical filter, the above-described mercury lamp and NUA filter (passing wavelength of 370 to 380 nm) were used, and the light passed through the NUA filter was collected by an objective lens having a 100× magnification and directed to the back surface of chip body 102.

Using the fabricated chip, fluorescent observation was conducted. For the fluorescent observation, the above-described halogen lamp and Cy5 filter were used as the light source and the optical filter, respectively, and the light passed through the Cy5 filter was collected by an objective lens of 20× magnification and directed to the back surface of the chip. The fluorescent light emitted from the chip was collected by using an objective lens of 20× magnification, and monitored by a CCD camera.

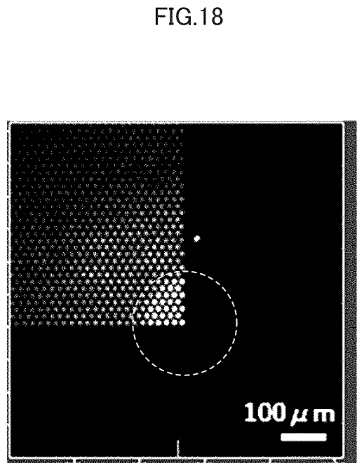

FIG. 18 shows an image picked-up by the CCD camera. Referring to FIG. 18, the circle (that is, white dotted line) shown at the center represents a border of an area irradiated with light to promote photoreaction at the time of manufacturing the chip. In FIG. 17, the circle in a broken line shown at the center corresponds to the circle at the center of FIG. 18. In FIG. 18, it can be seen that fluorescent light is hardly observed from the areas where plasmon-generating areas 104 are not formed. In the plasmon-generating area 104, because of the plasmon-enhanced electric field, fluorescent light can be observed. It can be seen that the fluorescent intensity from the plasmon-generating areas 104 formed inside the circle in the broken line (see FIG. 17) is higher than that from the plasmon-generating areas 104 formed in the surrounding area. Thus, it was confirmed that by light irradiation to promote photoreaction during manufacturing of the chip, bonding of Cy5-maleimide to the plasmon-generating area 104 was promoted.

In order to confirm the effect of light irradiation to promote photoreaction during manufacturing of the chip, quantitative evaluation was conducted. Table 1 shows fluorescent intensity measured in four areas, that is, Birr, Bout, Firr and Fout of FIG. 17.

TABLE 1

| Area | Fluorescent Intensity (relarive value) |
|---|---|
| Birr | 444.2 |
| Bout | 104.9 |
| Firr | 31.5 |
| Fout | 24.2 |

Using the measured values shown in Table 1, degree of enhanced fluorescence Ef and rate of promotion of chemical reaction Rp were calculated in accordance with the equations below. For convenience, the measured values in respective areas are denoted by the signs representing the areas.

$$Ef = Bout / Fout \qquad ( Equation 1 )$$

$$Rp = ( Birr - Bout ) / ( Firr - Fout ) \times 1 / Ef \qquad ( Equation 2 )$$

The areas Fout and Bout are not irradiated with light for promoting photoreaction and, therefore, Ef represents the effect of fluorescence enhancement attained by the plasmon-generating area 104. The area Birr was irradiated with light for promoting photoreaction while the area Bout was not irradiated with light for promoting photoreaction. Therefore, Birr-Bout includes the effect of photoreaction promotion and the effect of fluorescence enhancement. On the other hand, Firr-Fout represents only the effect of promoting photoreaction. Therefore, by dividing (Birr-Bout)/(Firr-Fout) by Ef as shown by the Equation 2 above, it is possible to evaluate the effect of promoting chemical reaction.

Using the values shown in Table 1, we obtain the degree of fluorescence enhancement Ef=about 4.3 and rate of chemical reaction promotion Rp=about 10.8. Namely, by light irradiation to promote photoreaction, it is considered that Cy5-maleimide can be bonded in a concentrated manner to the plasmon-generating areas 104 at about ten times higher density than to the area surrounding the plasmon-generating areas 104. Therefore, the detection sensitivity of fluorescent observation is remarkably improved to about 46.5 times higher (Rp×Ef) by the synergetic effect of promoted photoreaction and enhanced fluorescence. This and the results of comparative experiment described later as Example 2 clearly indicate that the present invention is very effective.

In an actual sensing chip, the plasmon-generating areas 104 in the area Birr in FIG. 17 will be formed entirely over the surface. Specifically, a sensing chip not including the areas Bout and Fout shown in FIG. 17 and including the areas corresponding to Birr and Firr (area on which plasmon-generating areas 104 are not formed) is possibly formed. Performance of such a chip can be evaluated by measuring fluorescent intensity in the areas Birr and Firr. Using the values of Table 1, Birr/Firr=about 13.9. While this value (Birr/Firr) may vary depending on the conditions of light irradiation for promoting photoreaction (including structural conditions of plasmon-generating areas 104), since Cy5-maleimide is coupled concentratedly in area Birr, it is expected that a value larger than the degree of fluorescence enhancement Ef (Ef=about 4.3 according to the values of Table 1) can be realized.

### Example 2

As a comparative example, using the same chip body as Example 1, a chip was fabricated through the same process but without performing the step of UV light irradiation to enhance photoreaction, and fluorescent observation was conducted. Specifically, as in Example 1, to the chip body 102 on which plasmon-generating areas 104 were formed, APTES (see FIG. 5) and o-Methylbenzaldehydes (see FIG. 6) were introduced, so as to cause coupling as shown in FIG. 7. In this state, without promoting photoreaction (without UV irradiation at the back surface of chip body 102), 3.12 nM of Cy5-maleimide was introduced, left standing still for a prescribed time period, and then rinsed to form a chip. Using the thus fabricated chip, fluorescent observation was conducted in the same manner as in Example 1. The results are shown in FIG. 2.

TABLE 2

| Area | Fluorescent Intensity (relarive value) |
|---|---|
| Birr | 92.2 |
| Bout | 87.2 |
| Firr | 19.4 |
| Fout | 18.4 |

Using the measured values shown in Table 2, degree of enhanced fluorescence Ef and rate of promotion of chemical reaction Rp were calculated in accordance with Equations 1 and 2 above, which were Ef=about 4.7 and Rp=about 1. Rp=about 1 indicates that without UV irradiation, photoreaction was not promoted. Therefore, Ef=about 4.7 indicates that fluorescent intensity was enhanced only by the coupling of non-specific absorption. The effectiveness of the present invention shown in Example 1 can be understood from these results.

### Example 3

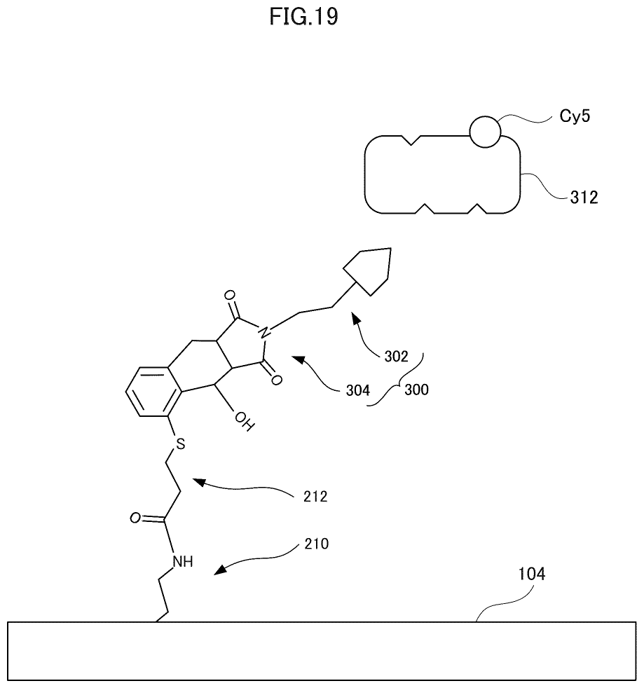

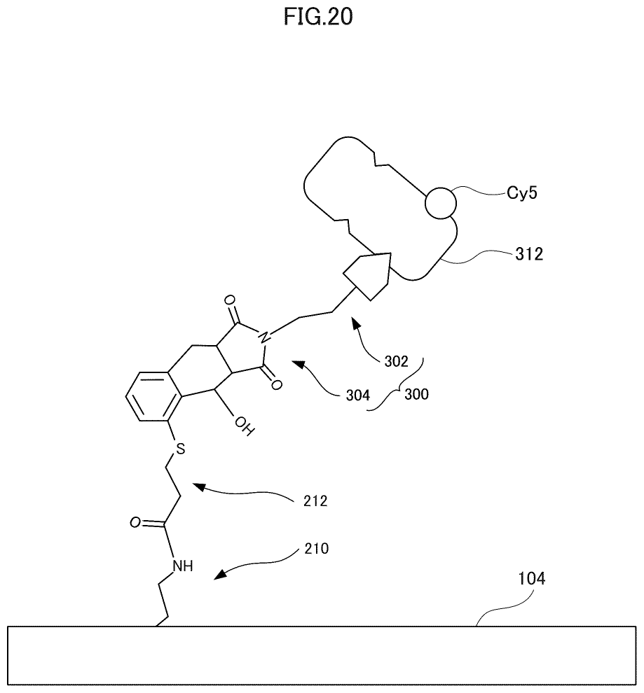

Using the chip fabricated in the same manner as Example 1, an experiment was done to confirm that target substance could be captured by the capturing molecules bonded to the chip. Specifically, the chip body (see FIG. 17) having the same structure, the same material and the same size as Example 1 was prepared. As in Example 1, APTES (see FIG. 5) was coupled to the chip body, and thereafter o-Methylbenzaldehydes (see FIG. 6) as photoreaction compound was introduced and coupled as shown in FIG. 7. Thereafter, the central portion of chip body (corresponding to the circle in dotted line in FIG. 17) was irradiated with UV light for 30 seconds to activate photoreaction compound, and in this state, biotin-maleimide prepared to about 1 μM was introduced and maintained, so that biotin as capturing molecules was bonded to the chip. Thus, referring to FIG. 19, a state in which the compound of APTES 210, benzaldehyde 212 and biotin-maleimide 300 is bonded to the plasmon-generating area 104 was attained. Of biotin portion 302 and maleimide portion 304 forming biotin-maleimide 300, biotin portion 302 functions as the capturing molecule. Thereafter, to the chip having biotin portion 302 bonded, protein fluorescent-labeled with Cy5 (specifically, streptavidin), specifically, Cy5-streptavidin 312 prepared in each of two types, that is, about 10 nM and about 1 nM, was introduced as the target substance (see FIG. 19). Thus, referring to FIG. 20, by the interaction of biotin and avidin, a combined body in which biotin portion 302 was coupled to Cy5-streptavidin 312, was formed. Specifically, a state was realized in which Cy5-streptavidin 312 was coupled to plasmon-generating area 104.

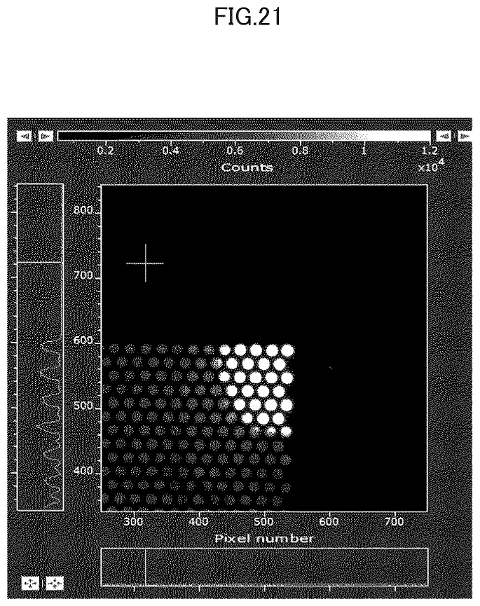

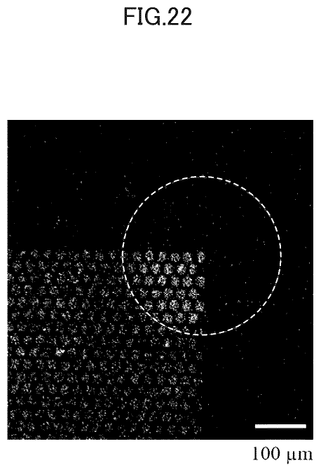

Using the thus fabricated chip, fluorescent observation was conducted as in Example 1. FIGS. 21 and 22 show images taken by a CCD camera. FIGS. 21 and 22 correspond to the chips fabricated by introducing Cy5-streptavidin 312 prepared to 10 nM and about 1 nM, respectively. A bar at the lower right corner of FIG. 22 indicates the length of 100 μm. Both in FIGS. 21 and 22, fluorescence is hardly observed from areas where plasmon-generating areas are not formed. In the plasmon-generating areas, fluorescence can be observed, because of the plasmon-enhanced electric field. It can be seen that the intensity of fluorescence from the plasmon-generating area at the center of each chip is higher than that from the surrounding plasmon-generating areas. This means that at the central portion of chip (that is, the area irradiated with UV light to promote photoreaction), much more Cy5-streptavidin is bonded than in the areas outer than the central portion. In other words, at the center of the chip, larger amount of biotin as the capturing molecules is bonded than in the areas outer than the center. Comparing FIGS. 21 and 22, at the central portion of the chips, fluorescent intensities depending on the concentration of introduced Cy5-streptavidin can be observed. This means that biosensing has been effectively done by the present chips.

As in Example 1, regarding the chip fabricated by introducing Cy5-streptavidin 312 prepared to about 10 nM, using the measurements inside and outside of the area irradiated with UV light to promote photoreaction at the time of manufacturing the chip, degree of enhanced fluorescence Ef and rate of promotion of chemical reaction Rp were calculated in accordance with Equations 1 and 2 above. The results are Ef=15 and Rp=1.2. The detection sensitivity of fluorescent observation was remarkably improved by 18 times (Rp×Ef), by the synergetic effect of photoreaction promotion and fluorescence enhancement.

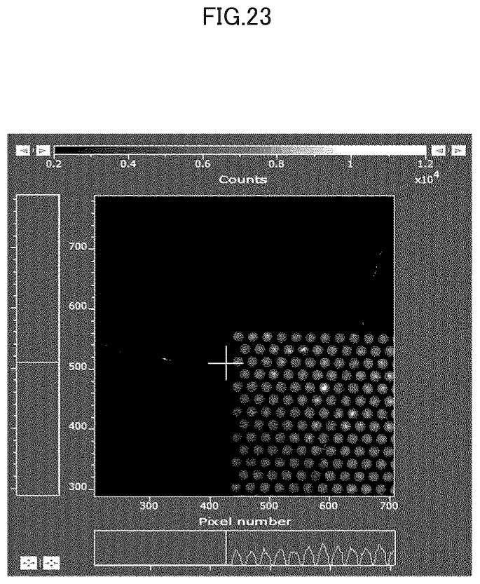

As a comparative experiment, using the same chip body as above, APTES was coupled to the chip body, o-Methylbenzaldehydes (see FIG. 6) was introduced, and without UV light irradiation for promoting photoreaction, biotin-maleimide was introduced. As a result, biotin as the capturing molecules was bonded to the chip by non-specific absorption. Using the fabricated chip, fluorescent observation was conducted as in Example 1. FIG. 23 shows an image taken by a CCD camera. In FIG. 23, fluorescence could be observed almost in uniformity in the plasmon-generating areas, because of the plasmon-enhanced electric field. From the comparison of FIGS. 21 and 23, effectiveness of UV light irradiation to promote photoreaction during chip manufacturing can be understood. Specifically, it becomes possible to bond the capturing molecules to the chip in a space-selective manner, and hence to improve detection accuracy of target substance by the capturing molecules.

### Example 4

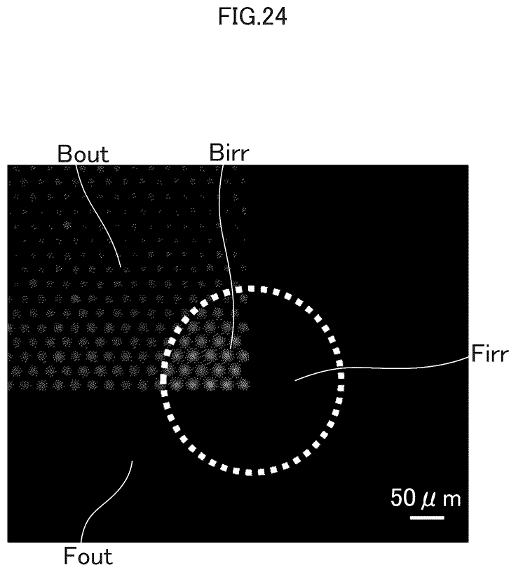

A chip interface was prepared by using visible light as the light for promoting photoreaction. Specifically, the chip body (see FIG. 17) of the same material and same size as Example 1 except for the structure at the central portion of each plasmon-generating area was prepared. The central portion has a recessed structure (that is, well structure) of ½ pitch size, and its recess/projection is reversed from that of FIG. 3. Using visible light (specifically, wavelength of 450 nm to 490 nm) in place of the UV light, as in Example 1, o-Methylbenzaldehydes (see FIG. 6) was coupled to the chip (see FIG. 7). For light irradiation, in the configuration shown in FIG. 12, a mercury lamp was used as light source 402, and a GFP filter (bandpass filter that passes wavelength of 450 nm to 490 nm) was used as optical filter 404 to generate the visible light (hereinafter referred to as GFP light). Thereafter, Cy5-maleimide prepared to 9.36 nM was introduced and bonded to the chip. Using the fabricated chip, fluorescent observation was conducted as in Example 1. FIG. 24 shows an image taken by a CCD camera. The bar on the lower right corner of FIG. 24 indicates the length of 50 μm. Fluorescent observation was done with the configuration shown in FIG. 12, a halogen lamp was used as light source 402 and objective lenses of 20× and 10× magnifications were used respectively as the first and second lenses 406 and 408. Fluorescent intensities measured for four areas Birr, Bout, Firr and Fout of FIG. 24 are shown in Table 3.

TABLE 3

| Area | Fluorescent Intensity (relarive value) |
|---|---|
| Birr | 121 |
| Bout | 103 |
| Firr | 8 |
| Fout | 8 |

Using the measured values shown in Table 3, degree of enhanced fluorescence Ef was calculated in accordance with Equation 1 above, the result was Ef=12.9. The rate of promotion of chemical reaction Rp to be calculated in accordance with Equation 2 above cannot be obtained, since the values of Firr and Fout in Table 3 were both the same “8.” Specifically, when the chip was manufactured using GFP light as the light to promote photoreaction, the fluorescent intensity in areas other than the plasmon-generating area was of comparable level no matter whether GFP light was irradiated or not. From this result, it can be seen that using GFP light could suppress bonding of capturing molecules in areas other than the plasmon-generating area, suppressed, and the capturing molecules were bonded in spatially selective manner, that is, bonded only to the plasmon-generating areas. This is because the GFP light having the wavelength of 450 nm to 490 nm corresponds to the absorption end of o-Methylbenzaldehydes (see FIG. 6) used as the photoreaction compound and the photoreaction hardly occurred.

In contrast, when UV light was used, even in the areas other than the plasmon-generating area, florescent intensity of “31.5” was measured in the area (for example, Firr) irradiated with the UV light as shown in Table 1. This value is clearly higher than the fluorescent intensity “24.2” of the area (for example, Fout) not irradiated with the UV light. This means that even in the areas other than the plasmon-generating areas, photoreaction proceeds if irradiated with the UV light and capturing molecules are bonded to certain extent. In other words, bonding of capturing molecules cannot be sufficiently suppressed.

### Example 5

An experiment was conducted to confirm that by using visible light to promote photoreaction, it becomes possible to bond the capturing molecules to the central portion of each plasmon-generating area with a high density. Specifically, the chip body (see FIG. 17) of the same structure, same material and same size as that of Example 4 was prepared. The central portion of each plasmon-generating area has a recessed structure of ½ pitch size. As in Example 4, using GFP light (wavelength of 450 nm to 490 nm) for promoting photoreaction, o-Methylbenzaldehydes (see FIG. 6) was coupled to the chip. Thereafter, Cy5-maleimide prepared to 9.36 nM was introduced, and then rinsed with PBS (phosphate buffer solution). The thus fabricated chip will be referred to as E5-Chip.

As the first comparative example, the same chip body (see FIG. 17) as described above was prepared, and o-Methylbenzaldehydes (see FIG. 6) was introduced to the chip, without irradiation of any light to promote photoreaction. Cy5-maleimide was not introduced. Thus fabricated chip will be referred to as CE1-Chip. Further, as the second comparative example, the same chip body (see FIG. 17) as described above was prepared, and o-Methylbenzaldehydes (see FIG. 6) was introduced to the chip without irradiation of any light to promote photoreaction. Thereafter, Cy5-maleimide prepared to 9.36 nM was introduced and then rinsed with PBS. Thus fabricated chip will be referred to as CE2-Chip.

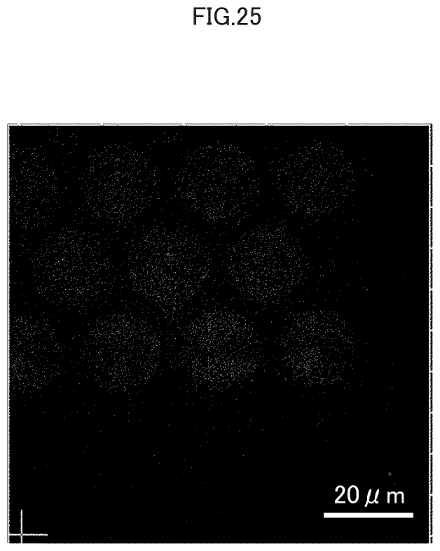

Using the three chips fabricated in the above-described manner, fluorescent observation was conducted as in Example 1. FIGS. 25 to 27 show images taken by a CCD camera. FIGS. 25 to 27 are images related to the CE1-Chip, CE2-Chip and E5-Chip, respectively, showing images of corresponding areas. The bar at the lower right corner of each of these figures indicates the length of 20 μm. Fluorescent observation was done with the configuration shown in FIG. 12, where a mercury lamp was used as light source 402, and objective lenses having 20× and 100× magnifications, respectively, were used as the first and second lenses 406 and 408.

In the fluorescence image of FIG. 25, plasmon-generating area can be faintly recognized. As to measurement values of CE1-Chip, fluorescent intensity B (BKG) of the plasmon-generating area and the fluorescent intensity F (BKG) in areas other than the plasmon-generating area were respectively “540” and “523.” The measurement values, including the values that will be shown below, are relative values represented based on the same standard. “BKG” represents background, and B (BKG) and F (BKG) correspond to levels of background noise inside and outside of the plasmon-generating area, respectively.

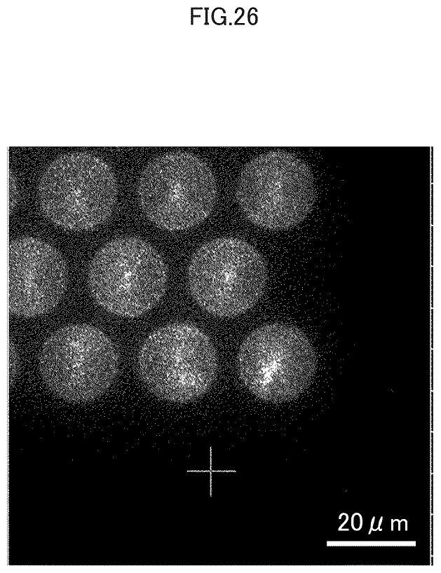

In the fluorescence image of FIG. 26, plasmon-generating area are clearly more recognizable than the fluorescence image of FIG. 25, and it can be seen that in each plasmon-generating area, the fluorescent intensity at the central portion tends to be higher than the fluorescent intensity in the periphery. In CE2-Chip, fluorescent intensity Bc (without irradiation) at the central portion of the plasmon-generating area, fluorescent intensity Be (without irradiation) at the periphery of the plasmon-generating area and the fluorescent intensity F (without irradiation) outside of the plasmon-generating area were “760,” “670” and “543,” respectively. Here, “without irradiation” indicates that irradiation of light for promoting photoreaction was not used.

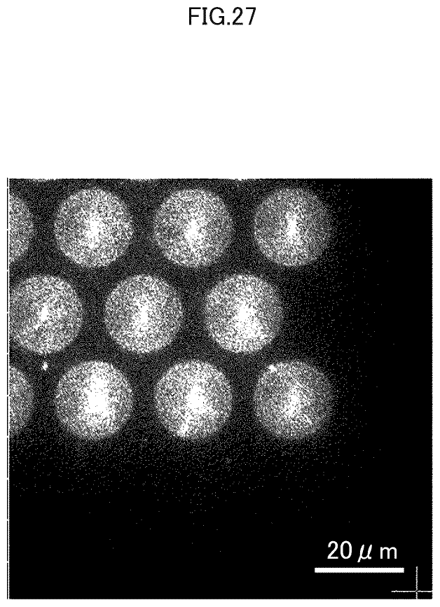

In the fluorescence image of FIG. 27, as compared with the image of FIG. 26, it can be seen that the fluorescent intensity in the plasmon-generating area is clearly increased. Further, it can be seen that in each plasmon-generating area, the fluorescent intensity at the central portion is clearly higher than the fluorescent intensity in the periphery. In E5-Chip, fluorescent intensity Bc (with irradiation) at the central portion of the plasmon-generating area, fluorescent intensity Be (with irradiation) at the periphery of the plasmon-generating area and the fluorescent intensity F (with irradiation) outside of the plasmon-generating area were “890,” “710” and “543,” respectively. Here, “with irradiation” indicates that irradiation of light for promoting photoreaction was conducted.

Using the measurement values above, it is possible to evaluate the fluorescent intensities with noise removed, based on values ΔB and ΔF obtained by subtracting the corresponding background noise (B(BKG) and F(BKG)). Specifically, in CE2-Chip, ΔBc (without irradiation)=220 (=760-540) at the central portion of the plasmon-generating area, and at the periphery, ΔBe (without irradiation)=130 (=670−540). Therefore, in CE2-Chip, at the central portion of the plasmon-generating area, fluorescent intensity 1.69 (=220/130) times higher than at the periphery was attained. CE2-Chip was not irradiated with light to promote photoreaction and, Cy5-maleimide is considered to be mainly bonded to the chip by non-specific absorption. Therefore, Cy5-maleimides bonded at the central area are considered to be in a similar number as those bonded at the periphery of the plasmon-generating area. Hence, the 1.69 times higher value results from the optical antenna effect at the time of fluorescent observation (that is, formation of enhanced electric field by localized plasmon at the central portion of the Bull's eye structure). It is noted that outside of the plasmon-generating area, ΔF (without irradiation)=20 (=543−523).

Similarly, regarding E5-Chip, it is possible to evaluate the fluorescent intensity with the noise removed. Specifically, in E5-Chip, ΔBc (with irradiation)=350 (=890−540) at the central portion of the plasmon-generating area, and at the periphery, ΔBe (with irradiation)=170 (=710−540). Therefore, in E5-Chip, at the central portion of the plasmon-generating area, fluorescent intensity 2.05 times higher (=350/170) than at the periphery was attained. Further, outside of the plasmon-generating area, ΔF (with irradiation)=20 (=543−523).

In E5-Chip, in addition to Cy5-maleimides coupled by non-specific absorption, Cy5-maleimides coupled by photoreaction are included. The values of ΔBc (with irradiation) and ΔBe (with irradiation) of E5-Chip are respectively higher than ΔBc (without irradiation) and ΔBe (without irradiation) of CE2-Chip, because of the photoreaction. In order to evaluate the influence of photoreaction, difference R (=ΔB (with irradiation)−ΔB (without irradiation) in the measurement values of corresponding areas was calculated regarding to E5-Chip and CE2-Chip. Using the calculated values above, as the difference Rc (=ΔBc (with irradiation)−ΔBc (without irradiation)) between E5-Chip and CE2-Chip at the central portion of the plasmon-generating area, Rc=130 (=350−220) is obtained. As the difference Re (=ΔBe (with irradiation)−ΔBe (without irradiation)) between E5-Chip and CE2-Chip at the periphery of the plasmon-generating area, Re=40 (=170−130) is obtained. Therefore, Rc/Re=3.25 (=130/40). Rc/Re represents, regarding Cy5-maleimides bonded by photoreaction, the ratio of fluorescent intensity at the central portion of the plasmon-generating area to the fluorescent intensity at the periphery. Specifically, as regards E5-Chip, it can be understood that by the capturing molecules bonded to the chip by photoreaction, 3.25 times higher fluorescent intensity was observed at the central portion of the plasmon-generating area than at the periphery. The magnification of “3.25” at the central portion also includes the influence of optical antenna effect at the time of fluorescent observation as described before and, therefore, by dividing it by the magnification “1.69” at the central portion of CE2-Chip described above, the influence of optical antenna effect in fluorescent observation can be removed. The calculated value is about 1.9 (=3.25/1.69). Namely, by the irradiation of light to promote photoreaction, 1.9 times larger number of capturing molecules than the periphery were bonded at the central portion of the plasmon-generating area. Specifically, it was confirmed that by using visible light as the light for promoting photoreaction, the capturing molecules could be bonded with a high density at the central portion of each plasmon-generating area.

The embodiments as have been described here are mere examples and should not be interpreted as restrictive. The scope of the present invention is determined by each of the claims with appropriate consideration of the written description of the embodiments and embraces modifications within the meaning of, and equivalent to, the languages in the claims.

### REFERENCE SIGNS LIST

100 sensing chip
102 chip body
104 plasmon-generating area
106, 180, 190 base substrate
110 first adhesive layer
112 metal layer
114 second adhesive layer
116 quench-suppressing layer
182, 192 projection
184, 194 recessed portion
200 coupled compound
202 capturing molecule
210 APTES
212 benzaldehyde
214 N-succinimidyl-3-maleimidepropionate
300 biotin-maleimide
302 biotin portion
304 maleimide portion
312 Cy5-streptavidin
400 measuring device
402 light source
404 optical filter
406 1st lens
408 2nd lens
410 camera
Birr, Bout, Firr, Fout areas
L1 period
L2, ϕ diameter

1: A sensing chip, comprising:
a substrate having a plasmon-generating area for generating propagating plasmon; and
a plurality of capturing molecules for capturing a target substance; wherein
said plurality of capturing molecules is bonded, by a compound including a photoreaction compound, to said plasmon-generating area at a higher density than to an area surrounding said plasmon-generating area.

2: The sensing chip according to claim 1, wherein
said plasmon-generating area includes a central portion of the plasmon-generating area and a surrounding area outside said central portion; and
said plurality of capturing molecules is bonded to said central portion at a higher density than to said surrounding area.

3: The sensing chip according to claim 1, wherein said plasmon-generating area has a concentric periodic structure of projections and recesses.

4: The sensing chip according to claim 3, wherein
a prescribed portion including the center of said concentric circles has a projected or recessed shape, and
said prescribed portion is a circle of which diameter is at most one period of the structure of projections and recesses.

5: The sensing chip according to claim 1, wherein
said capturing molecule includes biotin;
said photoreaction compound includes a compound represented by general formula (1) or (2) below or TFPA-PEG3-Biotin; and
said biotin is bonded to said plasmon-generating area by a compound of maleimide, said photoreaction compound and 3-Aminopropyl triethoxysilane: 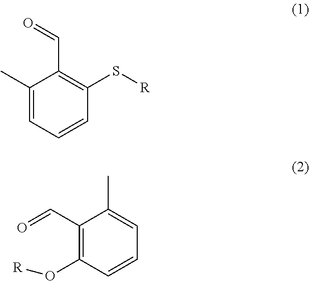
wherein
in the general formulae (1) and (2), R is any of the following compounds A1 to A11

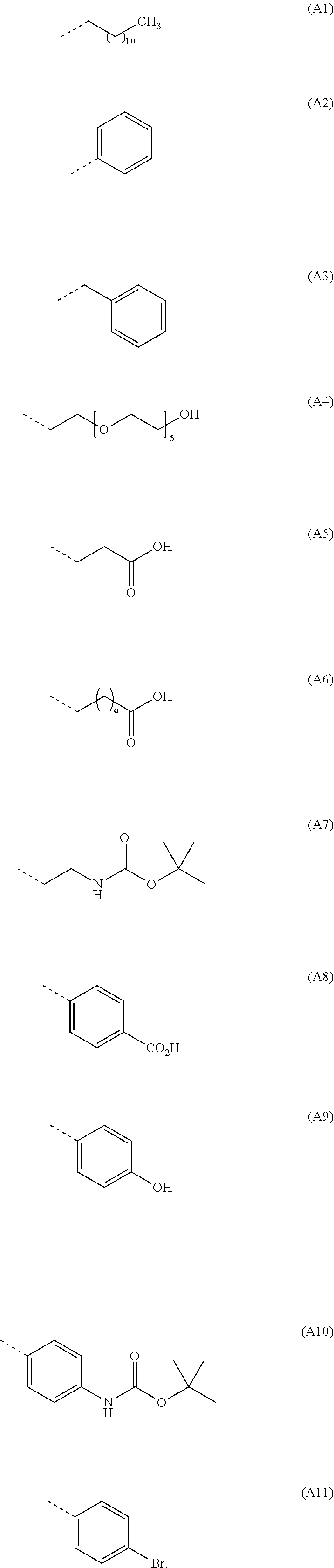

6: A method of manufacturing a sensing chip, comprising:
the first step of introducing a photoreaction compound bonded with capturing molecules for capturing a target substance, to a substrate having a plasmon-generating area; and
the second step of irradiating light at a back surface of said substrate after execution of said first step; wherein
at said second step, photoreaction of said photoreaction compound is promoted by plasmon-enhanced electric field, to have said capturing molecules bonded to said plasmon-generating area.

7: A method of manufacturing a sensing chip, comprising:
the first step of introducing capturing molecules for capturing a target substance to a substrate having a plasmon-generating area and having a photoreaction compound coupled; and
the second step of irradiating light at a back surface of said substrate after execution of said first step; wherein
at said second step, photoreaction of said photoreaction compound is promoted by plasmon-enhanced electric field, to have said capturing molecules bonded to said plasmon-generating area.

8: The method of manufacturing a sensing chip according to claim 6, wherein said light irradiated at said second step has a wavelength of at least 300 nm and at most 550 nm, or at least 600 nm and at most 1100 nm.

9: The method of manufacturing a sensing chip according to claim 6, wherein
said photoreaction compound includes a compound represented by general formula (1) or (2) below, or TFPA-PEG3-Biotin; 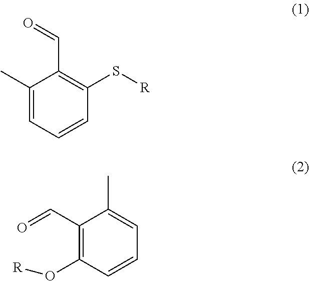
wherein
in the general formulae (1) and (2), R is any of the following compounds A1 to A11

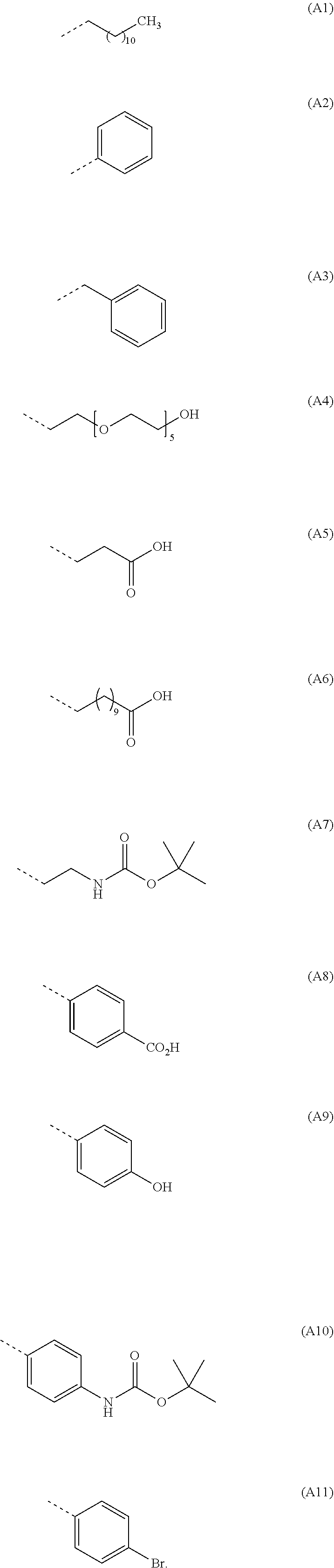

10: The method of manufacturing a sensing chip according to claim 9, wherein said light irradiated at said second step has a wavelength of at least 450 nm and at most 490 nm.

11: A sensing kit, comprising:
a substrate having a plasmon-generating area; and
a photoreaction compound; wherein
by introducing said photoreaction compound and a plurality of capturing molecules for capturing a target substance to said substrate and irradiating light at the back surface of said substrate, photoreaction of said photoreaction compound is promoted by a plasmon-enhanced electric field, and said photoreaction compound causes said plurality of capturing molecules to be bonded to said plasmon-generating area at a higher density than to an area surrounding said plasmon-generating area.

12: A measuring method, comprising:
the first step of introducing, to the sensing chip according to claim 1, said target substance to which said fluorescent substance is coupled; and
the second step of irradiating said sensing chip at its back surface with light after execution of said first step, and measuring fluorescent light emitted from said fluorescent substance by a plasmon-enhanced electric field, from the front surface of said sensing chip.

13: A measuring device, comprising:
a light source; and
a lens for collecting light from said light source; wherein
in a state where a photoreaction compound bonded with capturing molecules for capturing a target substance is introduced to a substrate having a plasmon-generating area, said substrate is irradiated at its back surface with the light collected by said lens, whereby photoreaction of said photoreaction compound is promoted by a plasmon-enhanced electric field and said capturing molecules are bonded to said plasmon-generating area;
said device further comprising
a measuring unit for measuring fluorescent light emitted from a fluorescent substance by a plasmon-enhanced electric field, after irradiating the substrate at its back surface with the light collected by the lens in a state where the target substance containing the fluorescent substance is introduced to the substrate having the capturing molecules bonded to the plasmon-generating area.
